(19) United States
(12) Patent Application Publication
(10) Pub. No.: US 2008/0102144 A1
(43) Pub. Date: May 1, 2008

(54) FAT BETA-OXIDATION ENHANCING AND CARBOHYDRATE ABSORPTION INHIBITION SUPPLEMENT

(75) Inventors: Ken Clement, Mississauga (CA); Marvin A. Heuer, Mississauga (CA); Shan Chaudhuri, Mississauga (CA)

Correspondence Address: TORYS LLP, 79 WELLINGTON ST. WEST, SUITE 3000, TORONTO, ON, M5K 1N2, US

(73) Assignee: IOMEDIX DEVELOPMENT INTERNATIONAL SRL, St. Michael (BB)

(21) Appl. No.: 11/867,481

(22) Filed: Oct. 4, 2007

Related U.S. Application Data
(60) Provisional application No. 60/863,209, filed on Oct. 27, 2006.

(51) Int. Cl. A61K 36/00; A61P 9/00

(52) U.S. Cl. 424/776

(57) ABSTRACT

The present invention relates to a method for increasing the β-oxidation of fatty acids while substantially simultaneously maintaining blood glucose concentration through the inhibition of carbohydrate absorption. Furthermore, the present invention additionally provides a composition for increasing the β-oxidation of fatty acids while substantially simultaneously maintaining blood glucose concentration through the inhibition of carbohydrate absorption comprising an Extract of Green Coffee Bean, and an Extract of Salacia oblonga. The composition may further comprise diacylglycerol.

### RELATED APPLICATIONS

The present application is related to and claims benefit of priority to U.S. Provisional Application No. 60/863,209 entitled “Fat β-oxidation Enhancing and Carbohydrate Absorption Inhibition Supplement” filed Oct. 27, 2006, the disclosure of which is hereby fully incorporated by reference.

### FIELD OF THE INVENTION

The present invention relates to a composition and method for simultaneously promoting fat β-oxidation and decreasing the absorption of carbohydrates by an individual resulting in maintenance of blood glucose levels following consumption of food.

### BACKGROUND

The main sources of fatty acids for oxidation, and thus energy, are cellular stores primarily in the form of triacylgylcerols contained within adipose tissue. In response to energy demands or stimulation through β-adrenergic signaling, the stored triacylgylcerols are mobilized for use in peripheral tissues. The release of this metabolic energy is continued by a series of cascading proteins resulting in the activation of hormone-sensitive lipase (HSL). As previously mentioned, the stimulation to initiate this cascade in adipocytes is typically a result of β-adrenergic signaling; however, it may also from glucagon, or β-corticotropin. The G-protein-linked receptors for these factors, upon binding, induce the activation of adenylate cyclase, resulting in an increase in the signaling molecule cyclic adenosine monophosphate (cAMP), leading to the activation of the signaling protein PKA. The activation of PKA in turn activates HSL inducing the release of fatty acids from carbons 1 or 3 of the triacylgylcerols. Together with HSL, diacylglycerol lipase catabolizes diacylglycerol to produce monoacylglycerol which is the substrate for monoacylglycerol lipase. The action of these three enzymes on their respective substrates results in release of 3 moles of fatty acid and one mole of glycerol. The resultant fatty acids then diffuse from adipose cells and combine with albumin in the blood for transport to tissues where energy is required.

In order that a free fatty acid is catabolized, it must first enter the mitochondria—the site of β-oxidation in a cell—such that β-oxidation may take place to produce energy. Since the oxidation of fatty acids is compartmentalized to the mitochondrion, the fatty acid must first be modified such that enzymes carnitine palmitoyltransferase 1 and 2 can transport the fatty acid to the border of the mitochondrion. In an unmodified form, fatty acids and their CoA derivatives are incapable of crossing the inner mitochondria membrane. Carnitine is the carrier molecule for the transport system of fatty acids into the mitochondrion, which is synthesized in humans from the amino acids lysine and methionine. Moreover, due to the energy demands of muscle tissue, carnitine is found in high concentration in muscle.

Upon entering a cell, a fatty acid must be altered to an activated state. This activated state is accomplished by fatty acid acyl-CoA synthetase, through the expenditure of ATP, linking a fatty acid and CoA. The activated fatty acid is covalently linked to carnitine by carnitine palmitoyltransferase 1, at the border of the mitochondrial membrane on the cytoplasmic side. This then allows the fatty acid, bonded to carnitine to enter the inner membrane of the mitochondrion wherein carnitine palmitoyltransferase 2 is located on the inner face of the inner membrane. Carnitine palmitoyltransferase 2 then releases fatty acid CoA and carnitine in to the matrix where it can be oxidized to form energy.

Deficiencies in carnitine palmitoyltransferases have been noted in medicine. A deficiency in carnitine palmitoyltransferase 1 has been shown to primarily affect the liver and result in a reduction in fatty acid oxidation and lead to ketogenesis. Deficiencies in carnitine palmitoyltransferase 2 have been shown to result in recurrent muscle fatigue and pain following strenuous exercise. These observations are the result of a lack of energy contained within those molecules entering the mitochondria for conversion to form adenosine triphosphate. Due to the need of transportation into the mitochondria, the activity of the carnitine palmitoyltransferases forms the basis for a rate-limiting step in the β-oxidation of fatty acids. The β-oxidation of fatty acids results a reduction of fat and an increase in produced energy.

The opposite mechanism of fat reduction and energy production from fatty acid is the production of fatty acids. Fatty acids can be stored in tissues, mainly adipose tissue until they are required for energy production.

In contrast to the β-oxidation of fatty acids occurring in the mitochondria, fatty acid synthesis occurs in the cytoplasm of a cells. Carbohydrates are enzymatically broken down to form glucose which then in turn forms pyruvate in the mitochondria followed by Acetyl CoA. Since fatty acid synthesis occurs in the cytoplasm it must first be converted to citrate in order to pass through the membrane of the mitochondria into in the cytoplasm where citrate lyase then converts it back to Acetyl CoA. This newly formed acetyl CoA begins the process of fatty acid synthesis whereby the CoA portion is lost as it joins the acyl carrier protein (ACP). The condensing enzyme (CE) portion of the complex also attaches a malonyl group. Through a series of condensation reactions in the fatty acid synthesis system, palmitate is formed which can be elongated to longer chain acids. In the liver, these fatty acids can be converted into triglycerides and transported to adipose tissue for storage.

If more glucose or carbohydrates are taken into the body than are required after muscle and liver glycogen stores are saturated, the excess is not excreted, but converted into fatty acids. The rate of fatty acid synthesis thus can be influenced by diet. In a diet wherein an individual consumes a high amount of simple carbohydrates, and low amounts of fat, lipogenic enzymes in the liver can be induced, leading to increased fatty acid and triglyceride synthesis, ultimately leading to the accrual of excess body fat by way of increased adipose tissue volume.

### SUMMARY OF THE INVENTION

The foregoing needs and other needs and objectives that will become apparent for the following description are achieved in the present invention, which comprises a method and composition for increasing the β-oxidation of fatty acids while simultaneously maintaining blood glucose concentration following food consumption. The method, comprising the administration of a composition comprising an Extract of Green Coffee Bean, diacylglycerol, and an Extract of Salacia oblonga results in a net fat loss balance.

### DETAILED DESCRIPTION OF THE INVENTION

In the following description, for the purposes of explanations, numerous specific details are set forth in order to provide a thorough understanding of the present invention. It will be apparent, however, to one skilled in the art that the present invention may be practiced without these specific details.

As used herein, the term ‘nutritional composition’ includes dietary supplements, diet supplements, nutritional supplements, supplemental compositions and supplemental dietary compositions or those similarly envisioned and termed compositions not belonging to the conventional definition of pharmaceutical interventions as is known in the art. Furthermore, ‘nutritional compositions’ as disclosed herein belong to category of compositions having at least one physiological function when administered to a mammal by conventional routes of administration.

Alternatively, formulations and nutritional compositions belonging to the present invention may be considered to be nutraceuticals. As used herein, the term ‘nutraceutical’ is recognized and used in the art to describe a specific chemical compound or combination of compounds found in, organic matter for example, which may prevent, ameliorate or otherwise confer benefits against an undesirable condition. As is known in the art, the term ‘nutraceutical’ is used to refer any substance that is a food, a part of food, or an extract of food which is suitable for consumption by an individual and providing physiological benefit which may be medical or health-related. Furthermore, the term has been used to refer to a product isolated, extracted or purified from foods or naturally-derived material suitable for consumption by an individual and usually sold in medicinal forms, such as caplets, tablet, capsules, Soft-Gel™ caplets, gel-caps and the like, not associated with food. Extracts suitable for use in the present invention may be produced by extraction methods as are known and accepted in the art such as alcoholic extraction, aqueous extractions, carbon dioxide extractions, for example.

The present invention is directed towards a method for increasing the β-oxidation of fatty acids while simultaneously maintaining blood glucose concentration following food consumption through the inhibition of carbohydrate absorption. Furthermore, the present invention additionally provides a composition for increasing the β-oxidation of fatty acids while simultaneously maintaining blood glucose concentration through the inhibition of carbohydrate absorption comprising an Extract of Green Coffee Bean, diacylglycerol, and an Extract of Salacia oblongs.

It is understood by the inventors that in order for an individual to have a reduction in fat mass, a net fat loss must result. The inventors believe that this is achieved through the use of a composition which not only increases the β-oxidation of fat through increasing the activity of carnitine palmitoyltransferases, but also through the partial inhibition of the absorption of carbohydrates.

Carnitine palmitoyltransferase activity comprises a rate-limiting step in the increases the β-oxidation of fatty acids. The carnitine palmitoyltransferase enzymes are required in order that the fatty acids can be transported across the mitochondrial membranes to the inner matrix where the fatty acids may be oxidized to produce energy. In times when there is insufficient activity in the carnitine palmitoyltransferases to transport fatty acids for oxidation, the free fatty acid can reform into triglycerides which are again stored in adipose tissue. Therefore, in order to increase fat loss, more fatty acids must be oxidized than those available to form triglycerides for storage in adipose tissue.

Furthermore, fatty acids can be synthesized from glucose. Glucose is the final enzymatic product of carbohydrate digestion. If an individual's glucose or carbohydrate intake exceeds the body's energy needs following the saturation of muscle and liver glycogen requirements, glucose is converted to acetyl CoA. The acetyl CoA can then be used for fatty acid synthesis in the liver and for the storage of triglycerides in adipose tissue.

It is understood by the inventors that a concomitant increase in an individuals ability to oxidize fatty acids and reduction or maintenance in blood glucose levels will results in a net fat loss balance.

Green Coffee Bean Extract

In vivo experiments studying the effects of chlorogenic acid, a major component of green coffee bean extracts, have shown that it is able to arrest the proliferation of 3T3-preadipocyte cells in the G1 phase of development. According to the experiments of Hsu et al. in 2006, the addition of chlorogenic acid to 3T3-preadipocyte tissue cultures inhibited the proliferation in both and a time- and dose-dependant manner. Furthermore, they determined that at doses >100 μM, cell viability was affected and apoptosis was induced (Hsu C L, Huang S L, Yen G C. Inhibitory effect of phenolic acids on the proliferation of 3T3-L1 preadipocytes in relation to their antioxidant activity. J Agric Food Chem. 2006 Jun. 14; 54(12):4191-7). It is understood by the inventors that based on this data, chlorogenic acid administration in vivo would translate into an inhibition of adipocytes differentiation and proliferation, resulting in a net reduction in adipose tissue.

Interestingly, in addition to the inhibitory effects of chlorogenic acid on proliferation, Johnston et al. in 2003 showed that glucose absorption may also be inhibited by chlorogenic acid. In their 2003 study, they showed that both caffeinated and decaffeinated coffee drinks had a similar response in that they both attenuated the postprandial secretion of glucose-dependent insulinotropic polypeptide (GIP) compared to control beverages. The GIP response is determined the by the rate of glucose absorption, thus the data of Johnston et al. 2003, suggest that chlorogenic acid decreases the intestinal rate of glucose absorption. (Johnston K L, Clifford M N, Morgan L M. Coffee acutely modifies gastrointestinal hormone secretion and glucose tolerance in humans: glycemic effects of chlorogenic acid and caffeine. Am J Clin Nutr. 2003 October; 78(4):728-33). Chlorogenic acid has also been shown to selectively inhibit hepatic glucose-6-phosphate (G-6-P), the rate-limiting step in gluconeogenesis and decrease hepatic triglyceride levels in mice following 14 days of administration. (Shimoda H, Seki E, Aitani M. Inhibitory effect of green coffee bean extract on fat accumulation and body weight gain in mice. BMC Complement Altern Med. 2006 Mar. 17; 6:9). It has been shown that about 33% of orally administered chlorogenic acid is absorbed in the small intestine of humans (Olthof M R, Hollman P C, Katan M B. Chlorogenic acid and caffeic acid are absorbed in humans. J. Nutr. 2001 January; 131(1):66-71), it is understood by the inventors that chlorogenic acid, found in extracts of green coffee bean is suitable to not only inhibit adipose tissue proliferation, but that it also contributes to the inhibition of glucose absorption.

The group of Shimoda et al. in 2006 reported that although dietary intake was not reduced during their green coffee bean extract treatments, green coffee bean extract significantly suppressed body weight. Furthermore, both the epididymal and perirenal fat pad mass with found to be significantly reduced in the group treated with 0.05% green coffee bean extract in the diet. It was also noted that the rate-limiting enzyme, carnitine palmitoyltransferase, which catalyses the transport of fatty acids into the mitochondria for oxidation showed an increase in activity in a dose-dependent manner in the liver. This dose-dependent enhancement of carnitine palmitoyltransferase was seen at 0.5% and 1.0% in green coffee extract supplemented diets after 6 days. Chlorogenic acid administration alone, however, did not enhance the activity of carnitine palmitoyltransferase (Shimoda H, Seki E, Aitani M. Inhibitory effect of green coffee bean extract on fat accumulation and body weight gain in mice. BMC Complement Altern Med. 2006 Mar. 17; 6:9). Total serum triglyceride levels were also decreased by green coffee extract and caffeine groups, suggesting that fatty acids must be oxidized and not converted back to triglycerides.

An embodiment of the present invention comprises from about 0.01 mg to about 10.0 mg of an Extract of Green Coffee Bean acid per serving of nutritional composition. The preferred amount of Extract of Green Coffee Bean per serving of the nutritional composition comprises about 1.0 mg.

Diacylglycerol

Diacylglycerol, in animal studies it was shown an increase in the β-oxidation of fatty acids when compared against triacylglycerol as disclosed in the article of Rudkowska et al. 2005. Additionally, a significant increase in hepatic fat oxidation is seen after a single dose of diacylglycerol oil as compared to triacylglycerol. It was also noted that glucose-6-phophate dehydrogenase, malic enzyme, fatty acid synthase and fatty acid synthesis were all shown to be lower in rats fed diacylglycerol, whereas factors such as mitochondrial and peroxisome oxidation of palmtoyl-CoA in liver homogenates were increased (Rudkowska I, Roynette C E, Demonty I, Vanstone C A, Jew S, Jones P J. Diacylglycerol: efficacy and mechanism of action of an anti-obesity agent. Obes Res. 2005 November; 13(11):1864-76). An increase in the β-oxidation of fatty acids has also been seen in human studies with the administration of diacylglycerol as opposed to triacylglycerol, wherein the increase in β-oxidation was observed in the absence of an increase in energy expenditure. Furthermore, it has been proposed that diacylglycerol consumption may be associated with improved appetite control and energy balance during states of increased β-oxidation of fatty acids (Rudkowska I, Roynette C E, Demonty I, Vanstone C A, Jew S, Jones P J. Diacylglycerol: efficacy and mechanism of action of an anti-obesity agent. Obes Res. 2005 November; 13(11):1864-76).

A proposed mechanism of action of diacylglycerol for increasing the β-oxidation of fatty acids compared to triacylglycerol is that triacylglycerol is hydrolyzed by 1,3-lipases, resulting 1,2-diacylglycerol and 2,3-diacylglycerol. The resultant diacylglycerol compounds are then subjected to additional lipases leading to 2-monoglycerol and free fatty acids which can then cross the intestinal wall and be used for the construction of chylomicron triglycerides. On the other hand, diacylglycerol at equilibrium consists of about 70% 1,3-diacylglycerol and about 30% 1,2-diacylglycerol. The action of lipases on these compounds results in glycerol and free fatty acids, which may be less readily resynthesized to chylomicron triglycerides compared to triacylglycerol. Additionally, larger amounts of free fatty acids digested from diacylglycerol may be released into the portal circulation rather than being incorporated into chylomicrons, thus resulting in lower serum triglyceride levels. (Rudkowska I, Roynette C E, Demonty I, Vanstone C A, Jew S, Jones P J. Diacylglycerol: efficacy and mechanism of action of an anti-obesity agent. Obes Res. 2005 November; 13(11):1864-76). This increased fatty acid oxidation leads to increased satiety, and therefore an individual will not feel the need to ingest calories, thus leading to fat and weight loss.

In 16-week study, the effects of a diet with 1,3-diacylglycerol versus. triacylglycerol were monitored. There was no difference in the daily energy intakes, fat intakes and percent of fat consumed between the two groups. However, it is interesting to note that there was, at the conclusion of the study a significant decrease in body weight, body mass index and waist circumference in the diacylglycerol supplemented group. Additionally in the diacylglycerol group, total fat area as assessed by CT scan was significantly reduced (Nagao T, Watanabe H, Goto N, Onizawa K, Taguchi H, Matsuo N, Yasukawa T, Tsushima R, Shimasaki H, Itakura H. Dietary diacylglycerol suppresses accumulation of body fat compared to triacylglycerol in men in a double-blind controlled trial. J Nutr. 2000 April; 130(4):792-7).

An embodiment of the present invention comprises between from about 0.01 mg to about 10.0 mg of diacylglycerol per serving of the nutritional composition. The preferred amount of diacylglycerol per serving of the nutritional composition is about 1.0 mg.

Salacia oblonga Extract

Salacia oblonga extract is known to be an α-glucosidase inhibitor. Glucosidase inhibitors decrease the absorption of carbohydrates from the intestine, resulting in a slower and lower rise in blood sugar following the consumption of a meal. Carbohydrates must be broken down before they can be absorbed from food into simple sugars, such as glucose, by enzymes in the intestine. α-glucosidase is one of the enzymes involved in breaking down carbohydrates. Through the inhibition of this enzyme, carbohydrates are not broken down as efficiently and glucose absorption is thus delayed or at least partially prevented. Heacock et al., 2005, showed that compared to controls in non-diabetic adults a dose of 1000 mg of Salacia oblonga reduced serum glucose and insulin levels by 29% (p=0.01) 120 minutes following the ingestion of a study beverage consisting of 14 g of fat, 82 g of carbohydrates, and 20 g of protein (Heacock P M, Hertzler S R, Williams J A, Wolf B W. Effects of a medical food containing an herbal alpha-glucosidase inhibitor on postprandial glycemia and insulinemia in healthy adults. J Am Diet Assoc. 2005 January; 105(1):65-71). In a separate study, following the administration of a beverage the same as outlined above, it was determined that at 120 minutes following the simultaneous administration of 1000 mg of an extract of Salacia oblonga, plasma glucose was reduced relative to controls by 27% (p=0.035) for area under the curve measurements. The same study also determined under the above conditions that at times 120 and 180 minutes following the administration of the study beverage and Salacia oblonga extract there was a 35% and 36% (p<0.001) reduction in serum insulin levels compared to control (Collene A L, Hertzler S R, Williams J A, Wolf B W. Effects of a nutritional supplement containing Salacia oblonga extract and insulinogenic amino acids on postprandial glycemia, insulinemia, and breath hydrogen responses in healthy adults. Nutrition. 2005 July-August; 21 (7-8):848-54). These results suggest that the Salacia oblonga extract is effective in decreasing glycemia through its activity as an α-glucosidase inhibitor.

An embodiment of the present invention comprises between from about 0.001 mg to about 100 mg of an Extract of Salacia oblonga per serving of the nutritional composition. In an embodiment, the nutritional composition comprises from about 0.01 mg to about 10.0 mg of an Extract of Salacia oblonga per serving. In a further embodiment, the nutritional composition comprises about 1.0 mg of an Extract of Salacia oblonga per serving.

It is understood by the inventors that individuals seeking to reduce their bodily fat mass would benefit from a composition and accompanying method of use for said composition which inhibits the absorption of carbohydrates, thus maintaining blood glucose levels following food consumption and enhances the β-oxidation of fatty acids. The present invention provides a composition for the simultaneous delivery of compounds to enhance the β-oxidation of fatty acids and for the inhibition of carbohydrate absorption through the inhibition of α-glucosidase to maintain blood glucose levels following food consumption. By way of oral administration of the composition of the present invention, a method is provided to enhance the β-oxidation of fatty acids and maintain blood glucose levels following food consumption.

Additional embodiments of the present invention may also include portions of the composition as fine-milled ingredients. U.S. Non-Provisional patent application Ser. No. 11/709,526 entitled “Method for Increasing the Rate and Consistency of Bioavailability of Supplemental Dietary Ingredients” filed Feb. 21, 2007, which is herein fully incorporated by reference, discloses a method of increasing the rate of bioavailability following oral administration of components comprising supplemental dietary compositions by the process of particle-milling. For the purposes of the present invention, the terms micronization, milling, particle-milling, and fine-milling are used interchangeably, wherein they refer to a technology, process and end-products involved in or leading to a narrowing of particle size range and a concomitant reduction in the average particle size. For the purposes of the present invention, acceptable milled-particle sizes are in the range of from about 1 nanometer to about 500 microns.

Further to improving bioavailability, it is understood by the inventors that increased solubility resulting from fine-milling will lead to improvements in characteristics in which solubility and reduced particle size likely play a role.

Furthermore, additional embodiments of the present invention may be incorporated into specific controlled-release solid dosage forms. U.S. Non-Provisional patent application Ser. No. 11/709,525 entitled “Method for a Supplemental Dietary Composition Having a Multi-Phase Dissolution Profile” filed Feb. 21, 2007, which is herein fully incorporated by reference, discloses a method of achieving a solid oral dosage form with multiple dissolution characteristics for the release of active ingredients. Conventional oral dosage formulations are bound by the rate of dissolution of the unprocessed substance, thereby limiting the rate of bioavailability of the substance upon oral administration. This is particularly problematic for poorly-soluble compounds which have an inherently low rate of dissolution in that they may be excreted prior to first-pass.

It is herein understood that, due to the relationship between solubility and dissolution, the amount of a substance in solution at any given time is dependent upon both dissolution and solubility. Furthermore, it is understood by way of extension that increasing the rate of dissolution of a given substance acts to reduce the time to dissolution of a given solute or substance in a given solvent. However, the absolute solubility of said solute does not increase with infinite time. Thus, increasing the rate of dissolution of a substance will increase the amount of said substance in solution at earlier points in time, thus increasing the rate of bioavailability of said substance at earlier times upon oral administration.

The increase in the rate of bioavailability will allow better and quicker compound transfer to the systemic parts of the body.

Micronization is a technique which has been used as a method of sizing solid compounds to fine powders. Following a micronization process, compounds and more specifically poorly soluble compounds are transformed into fine powders which can then be transformed into suitable, stable and patient-compliant dosage forms. These forms, for the purposes of the present invention are derived for oral administration.

Micronization techniques offer an advantage over larger forms of compounds and poorly soluble compounds—following micronization, compounds have higher surface area to volume ratio. This provides for, as compared to physically coarse compounds, an ultrafine micronized powder that has a significantly increased total surface area. Mathematically, cross-sectional surface area increases with the square of the radius, while volume increases with the cube of the radius. Therefore, as a particle becomes smaller, the volume of the particle decreases at a faster rate than the surface area leading to an increase in the ratio of surface area to volume. By way of theoretical calculations, decreasing the size of a particle can increase its rate of dissolution via increasing the surface area to volume ratio. In the case of solubility, this increase in relative surface area allows for greater interaction with solvent. Additional embodiments of the present invention may employ a multi-phasic dissolution profile to provide a time-release mechanism. The fine-milling process may be employed in the processing of one or more of the ingredients of the present invention in the dosage forms of tablets, e.g., immediate-release film coated, modified-release and fast-dissolving; capsules, e.g., immediate-release and modified-release; liquid dispersions; powders; drink mixes, etc.

According to various embodiments of the present invention, the nutritional supplement may be consumed in any form. For instance, the dosage form of the nutritional supplement may be provided as, e.g., a powder beverage mix, a liquid beverage, a ready-to-eat bar or drink product, a capsule, a liquid capsule, a tablet, a caplet, or as a dietary gel. The preferred dosage form of the present invention is as a tablet.

Although the following examples illustrate the practice of the present invention in two of its embodiments, the examples should not be construed as limiting the scope of the invention. Other embodiments will be apparent to one of skill in the art from consideration of the specifications and example.

Extensions and Alternatives

In the foregoing specification, the invention has been described with specific embodiments thereof; however, it will be evident that various modifications and changes may be made thereto without departing from the broader spirit and scope of the invention.

### EXAMPLE

### Example 1

A nutritional composition is provided in the form of a tablet. Each serving of the nutritional composition comprises the following;
about 1.0 mg of and Extract of Green Coffee Bean, about 1.0 mg Diacylglycerol and about 1.0 mg of an Extract of Salacia Oblonga.
   As a method of enchancing β-oxidation of fatty acids, inhibiting carbohydrate absorption through the inhibition of α-glucosidase to maintain blood glucose levels following food consumption, the nutritional composition is administered to a mammal as required, preferably prior to a meal, once daily.

### Example 2

A nutritional composition is provided in the form of a tablet. Each serving of the nutritional composition comprises the following;
about 1.0 mg of fine-milled Extract of Green Coffee Bean, about 1.0 mg of fine-milled Diacylglycerol and about 1.0 mg of fine-milled Extract of Salacia oblonga.
   As a method of enchancing β-oxidation of fatty acids, inhibiting carbohydrate absorption through the inhibition of α-glucosidase to maintain blood glucose levels following food consumption, the nutritional composition is administered to a mammal as required, preferably prior to a meal, once daily.

What is claimed:

1. A composition for increasing the β-oxidation of fatty acids and substantially simultaneously maintaining blood glucose concentration comprising from about 0.01 mg to about 10.0 mg of an extract of Green Coffee Bean;
about 0.01 mg to about 10.0 mg of Diacylglycerol; and
about 0.01 mg to about 10.0 mg of an extract of Salacia oblonga.

2. The composition of claim 1, wherein the amount of an extract of Green Coffee Bean is about 1 mg;
the amount of Diacylglycerol is about 1 mg; and
the amount of extract of Salacia oblonga is about 1 mg.

3. The composition of claim 1, wherein at least a portion of the extract of Green Coffee Bean, and the extract of Salacia oblonga are fine-milled.

4. The composition of claim 1 wherein the extract of Green Coffee Bean is caffeine-free

5. The composition of claim 1 wherein the extract of Green Coffee Bean, the Diacylglycerol, and the extract of Salacia oblonga comprise a solid oral dosage form having a multi-phasic rate of dissolution.

6. The composition of claim 5 wherein said multi-phasic rate of dissolution comprises a first-phase and a second-phase; whereby said first-phase has a first rate of dissolution said second-phase has a second rate of dissolution.

7. The composition of claim 6, further comprising a third-phase, whereby said third-phase has a third rate of dissolution.

8. The composition of claim 6, wherein the multi-phasic rate of dissolution provides a time-release mechanism.

9. A method of increasing β-oxidation of fatty acids and substantially simultaneously maintaining blood glucose concentration in a mammal comprising at least the step of administrating to the mammal, a composition comprising from about 0.01 mg to about 10.0 mg an extract of Green Coffee Bean;
from about 0.01 mg to about 10.0 mg of Diacylglycerol; and
from about 0.01 mg to about 10.0 mg of an extract of Salacia oblonga.

10. The method of claim 9, wherein the composition is administered at least once daily.
